(19) United States
(12) Patent Application Publication
(10) Pub. No.: US 2025/0072955 A1
(43) Pub. Date: Mar. 6, 2025

(54) SYSTEMS AND METHODS FOR TREATING HEADACHE DISORDERS

(71) Applicant: Neurent Medical Limited, Oranmore (IE)

(72) Inventors: Adriona Kelly, Galway (IE); Steven Horan, Galway (IE); Aidan Loughrey, Galway (IE)

(21) Appl. No.: 18/825,146

(22) Filed: Sep. 5, 2024

Related U.S. Application Data
(60) Provisional application No. 63/536,557, filed on Sep. 5, 2023.

(51) Int. Cl. A61B 18/14; A61B 17/00; A61B 18/00; A61B 18/02

(52) U.S. Cl. CPC A61B 18/1485; A61B 18/02; A61B 2017/00867; A61B 2018/00327; A61B 2018/00434; A61B 2018/00577; A61B 2018/1475

(57) ABSTRACT

The invention generally relates to systems and methods for therapeutically modulating nerves in or associated with a sino-nasal region of a patient for the treatment of headache disorders.

### CROSS-REFERENCE TO RELATED APPLICATIONS

This application claims the benefit of, and priority to, U.S. Provisional Patent Application No. 63/536,557, filed Sep. 5, 2023, the content of which is incorporated by reference herein in its entirety.

### FIELD OF THE INVENTION

The invention generally relates systems and methods for treating medical conditions, and, more particularly, to systems and methods for therapeutically modulating nerves in a sino-nasal region of a patient for the treatment of headache disorders.

### BACKGROUND

Headaches are a very common condition that most people will experience many times during their lives. The main symptom of a headache is pain within one's head or face. Headache disorders, characterized by recurrent headache, are among the most common disorders of the nervous system. A headache itself is a painful and disabling feature of a small number of primary headache disorders, namely migraine, tension-type headache, and cluster headache. Headaches can also be caused, by or occur secondarily, to a list of other conditions, the most common of which is medication-overuse headache.

Headaches can range in frequency and severity of pain. Some individuals may experience headaches once or twice a year, while others may experience them more than 15 days a month. Some headaches may recur or last for weeks at a time. Pain can range from mild to disabling and may be accompanied by symptoms such as nausea or increased sensitivity to noise or light.

Globally, it has been estimated that prevalence among adults of current headache disorder (symptomatic at least once within the last year) is about 50%. About half to three quarters of adults aged 18-65 years in the world have had a headache within the last year and, among those individuals, 30% or more have reported migraines. Despite regional variations, headache disorders are a worldwide problem, affecting people of all ages, races, income levels and geographical areas.

### SUMMARY

The invention recognizes that a problem with current methods of treating headache disorders are either temporary or are not accurate and further fail to adequately treat the underlying cause, thereby failing to adequately address chronic headache disorders.

The invention solves these problems by providing systems and methods for treating headache disorders by providing, among other things, systems and methods for therapeutically modulating neural structures associated with headache disorders. For example, the present invention includes a treatment device including an end effector for delivering energy to target sites within the sino-nasal cavity that include, but are not limited to, anterior ethmoidal nerve (AEN) tissue, posterior nasal nerve (PNN) tissue, and sphenopalatine ganglion (SPG) nerve cells. For example, the energy delivered to target site(s) within the sino-nasal cavity is sufficient to therapeutically treat neural tissues of interest associated with the AEN tissue, PNN tissue, and/or SPG nerve cells. In particular, energy delivered to the target site(s) may be sufficient to therapeutically modulate or interrupt neural signals associated with AEN tissue, PNN tissue, and/or SPG nerve cells, thereby reducing the frequency and intensity of headache disorder-related systems, thereby treating headache disorders at their core.

The treatment device may include various forms of an end effector shaped and/or sized to be positioned within a sino-nasal cavity and relative to target sites associated with the AEN, PNN, and SPG. For example, in some embodiments, the end effector may generally include a single segment or multiple segments, each of which is comprises of one or more flexible support elements that include at least one energy delivering element for delivering treatment energy to the desired target site. The flexible support elements may include a specific geometry once deployed within the sino-nasal cavity to completement anatomy of a respective location, such that a given support element may contact and generally conform to a shape of a respective location, at which point energy may be delivered to the underlying tissue (i.e., neural tissue) at a target site.

The energy delivering elements may include electrodes or the like configured to apply electromagnetic neuromodulation energy (e.g., radiofrequency (RF) energy) to target sites. It should be noted that, in other embodiments, the end effector may include other energy delivery elements configured to provide therapeutic neuromodulation using various other modalities, such as cryotherapeutic cooling, ultrasound energy (e.g., high intensity focused ultrasound (“HIFU”) energy), microwave energy (e.g., via a microwave antenna), direct heating, high and/or low power laser energy, mechanical vibration, and/or optical power.

Once the end effector is delivered within the sino-nasal cavity and positioned relative to the desired target site(s) (i.e., relative to the AEN, PNN, and/or SPG), portions of the end effector (i.e., flexible support elements) can transition to a deployed state, having a specific shape and/or size corresponding to anatomical structures within the sino-nasal cavity and associated with target sites to undergo delivery of therapeutic energy for treatment of headache disorders. As such, once deployed, the energy delivery element(s) can be positioned at desired locations for focused application of energy to the underlying targeted neural tissue at the one or more target sites.

In one aspect, the present invention includes a method for treating a headache disorder of a patient. The method includes delivering treatment energy to one or more tissues at one or more target sites within a sino-nasal cavity of the patent to thereby cause multipoint interruption and/or multipoint modulation of one or more targeted neural tissues for the treatment of a headache disorder. The targeted neural tissue is associated with at least one of anterior ethmoidal nerve (AEN) tissue, posterior nasal nerve (PNN) tissue, and sphenopalatine ganglion (SPG) nerve cells.

The delivery of treatment energy can create targeted micro-lesions in targeted neural tissue to thereby interrupt associated neural signals from passing therethrough.

For example, in one embodiment, the targeted micro-lesions cause multiple points of interruption of postganglionic parasympathetic fibers and trigeminal afferent branches to thereby reduce the frequency and intensity of headache disorder-related symptoms. The sphenopalatine ganglion (SPG) has been implicated in many headache disorders. The SPG lies behind the middle turbinate in the pterygopalatine fossa and contains a large cluster of sympathetic, parasympathetic, and sensory neurons that lies within a small pyramidal space. It is thought that headaches and facial pain originate from the activation of parasympathetic pathways within SPG. Irritation of the SPG causes cerebral vasodilatation, increases cerebral blood flow, and in turn releases acetylcholine (which can activate nociceptive fibers), and further releases nitric oxide, which ultimately cause headaches. Accordingly, multipoint lesion creation in the SPG region could result in multipoint interruption of postganglionic parasympathetic fibers and trigeminal afferent branches and reduce the frequency and intensity of headache symptoms.

In another embodiment, the targeted micro-lesions cause multiple points of interruption of nociceptive C fibers to thereby reduce the frequency and intensity of headache disorder-related symptoms. The PNN is an accessible trigeminal branch of the SPG that innervates the nasal cavity along the turbinates and along the lateral wall. The PNN consists of trigeminal afferent branches and postganglionic parasympathetic fibers, which release neuropeptides such as calcitonin gene-related peptide (CGRP), substance P, as well as vasoactive intestinal peptide (which widens the blood vessels) upon stimulus and irritation. These neuropeptides, upon activation, can contribute significantly to pain and sensation and may contribute to symptoms such as headache and facial pain. Headaches/migraines usually accompany nasal symptoms. However, whether the symptoms occur due to migraine or vice versa is debatable. Despite this, it is believed that treatment of the pathologies of the nose will reduce frequency and intensity of headaches. Accordingly, multipoint lesion creation in the PNN region could result in multipoint interruption of nociceptive c fibers that cause sensation and pain, thus reduce the intensity and frequency of headaches and facial pain.

In another embodiment, the targeted micro-lesions cause multiple points of interruption of accessible trigeminal branches associated with PNN tissue to thereby reduce the frequency and intensity of headache disorder-related symptoms. Furthermore, the targeted micro-lesions cause multiple points of interruption of AEN tissue to thereby reduce the frequency and intensity of headache disorder-related symptoms.

The secretion of neuropeptides CGRP and substance P from the trigeminal nerve and VIP from parasympathetic fibers are involved in the pathophysiology of CRS. The neuropeptides are released upon stimulus/irritation of trigeminal afferent branches and parasympathetic fibers. Ethmoidal sinuses receive sensory and parasympathetic information from the trigeminal branches, including anterior ethmoid and posterior ethmoid nerves (from the nasociliary nerve), and posterior lateral superior nasal nerve (from the pterygopalatine ganglion). The ablation of these trigeminal branches could relieve some of the sino-nasal symptoms experienced with ethmoidal sinusitis. The posterior ethmoidal nerve is often an inaccessible nerve due to either being absent in the patient or being surrounded by important olfactory nerves (responsible for sense of smell). Accordingly, multipoint lesion creation in accessible trigeminal branches, such as the lateral superior posterior nasal branches (including along the middle meatus) and AEN may relieve some of these symptoms. Furthermore, the present invention is able to deliver precise levels of treatment energy such that energy is sufficient to create the targeted micro-lesions in the AEN tissue without damaging olfactory nerves to thereby preserve olfactory function.

Accordingly, the systems and methods of the present invention allow for accurate, minimally invasive, and localized application of energy to one or more target sites within the sino-nasal cavity to disrupt neural signals associated with nerves that play a role in headache disorders. By treating the underlying cause of headache disorders, the present invention is able to better address the symptoms without the inherent risks and drawbacks associated with pharmacotherapy and surgical treatments.

### BRIEF DESCRIPTION OF THE DRAWINGS

FIGS. 1A and 1B are diagrammatic illustrations of a system for treating a condition of a patient using a handheld device according to some embodiments of the present disclosure.

FIG. 2 is a diagrammatic illustration of the console coupled to the handheld device consistent with the present disclosure, further illustrating one embodiment of an end effector of the handheld device for delivering energy to tissue at one or more target sites.

FIG. 3 a cut-away side view illustrating the anatomy of a lateral sino-nasal wall.

FIG. 4 is a side view of one embodiment of a handheld device for providing therapeutic treatment consistent with the present disclosure.

FIGS. 5A-5F are various views of a multi-segment end effector consistent with the present disclosure.

FIG. 5A is an enlarged, perspective view of the multi-segment end effector illustrating the first (proximal) segment and second (distal) segment. FIG. 5B is an exploded, perspective view of the multi-segment end effector. FIG. 5C is an enlarged, top view of the multi-segment end effector. FIG. 5D is an enlarged, side view of the multi-segment end effector. FIG. 5E is an enlarged, front (proximal facing) view of the first (proximal) segment of the multi-segment end effector. FIG. 5F is an enlarged, front (proximal facing) view of the second (distal) segment of the multi-segment end effector.

FIGS. 6A and 6B are enlarged perspective views of the second (distal) segment of the multi-segment end effector including a flexible printed circuit board (PCB) assembly operably associated therewith.

FIGS. 7A and 7B are enlarged perspective views of another embodiment of an end effector consistent with the present disclosure, illustrating loop-shaped struts or leaflets that are generally extending back towards the shaft of the device when in an expanded, deployed state.

FIGS. 8 and 9 are side views showing single loop-shaped struts of an end effector consistent with the present disclosure that can be used for delivering energy to target tissue at target sites within the sino-nasal cavity.

FIG. 10 is a cut-away side view illustrating one approach for delivering an end effector to a target site within a sino-nasal region of a patient and subsequently delivering energy to one or more target tissues associated with AEN tissue for the treatment of headache disorder in accordance with embodiments of the present disclosure.

FIG. 11 is a cut-away side view illustrating another approach for delivering an end effector to a target site within a sino-nasal region of a patient and subsequently delivering energy to one or more target tissues associated with SPG nerve cells for the treatment of headache disorder in accordance with embodiments of the present disclosure.

### DETAILED DESCRIPTION

Headache, also known as cephalalgia, is the symptom of pain in the face, head, or neck. It can occur as a migraine, tension-type headache, or cluster headache, among others.

Headaches can occur as a result of many conditions. There are a number of different classification systems for headaches. The most well-recognized is that of the International Headache Society, which classifies it into more than 150 types of primary and secondary headaches. Causes of headaches may include dehydration; fatigue; sleep deprivation; stress; the effects of medications (overuse) and recreational drugs, including withdrawal; viral infections; loud noises; head injury; rapid ingestion of a very cold food or beverage; and dental or sinus issues (such as sinusitis).

A headache is one of the most commonly experienced of all physical discomforts. The treatment of a headache depends on the underlying cause, but commonly involves pain medication (especially in case of migraine or cluster headaches). Primary headache syndromes have many different possible treatments. In those with chronic headaches, the long term use of opioids appears to result in greater harm than benefit.

The invention recognizes that a problem with current methods of treating headache disorders are either temporary or are not accurate and further fail to adequately treat the underlying cause, thereby failing to adequately address chronic headache disorders.

The invention solves these problems by providing systems and methods for treating headache disorders by providing, among other things, systems and methods for therapeutically modulating neural structures associated with headache disorders. For example, the present invention includes a treatment device including an end effector for delivering energy to target sites within the sino-nasal cavity that include, but are not limited to, anterior ethmoidal nerve (AEN) tissue, posterior nasal nerve (PNN) tissue, and sphenopalatine ganglion (SPG) nerve cells. For example, the energy delivered to target site(s) within the sino-nasal cavity is sufficient to therapeutically treat neural tissues of interest associated with the AEN tissue, PNN tissue, and/or SPG nerve cells. In particular, energy delivered to the target site(s) may be sufficient to therapeutically modulate or interrupt neural signals associated with AEN tissue, PNN tissue, and/or SPG nerve cells, thereby reducing the frequency and intensity of headache disorder-related systems, thereby treating headache disorders at their core.

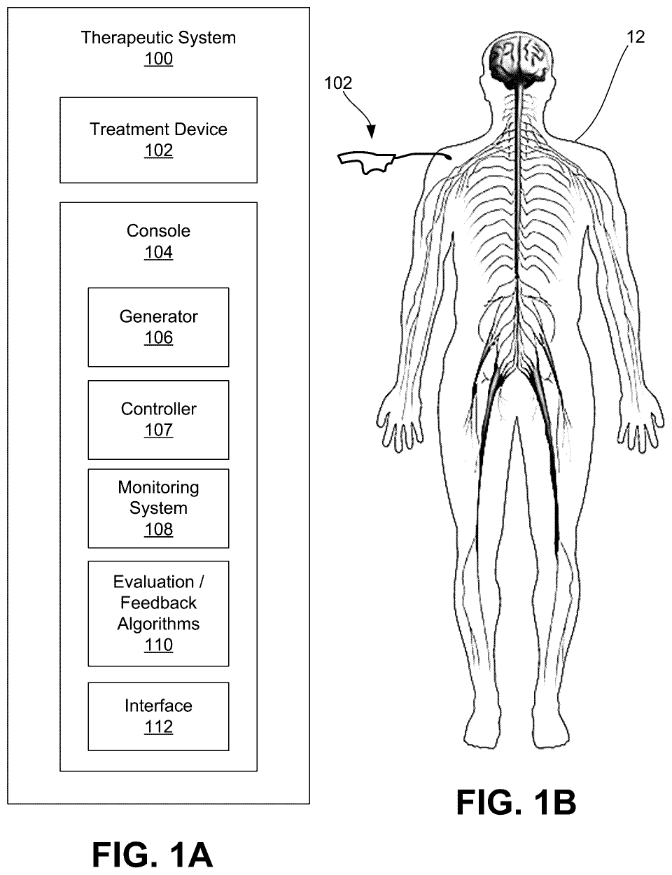

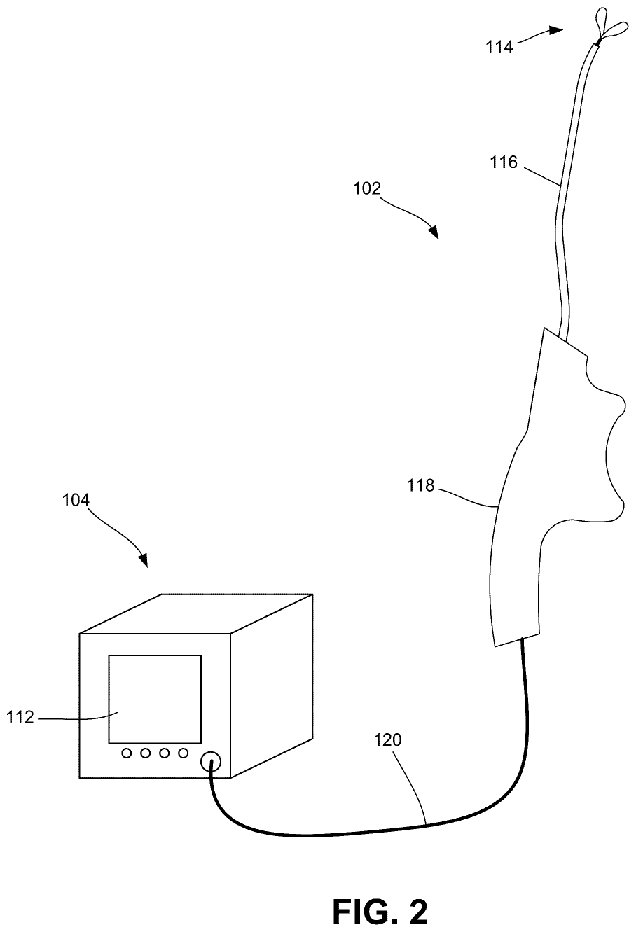

FIGS. 1A and 1B are diagrammatic illustrations of a therapeutic system 100 for treating a condition of a patient using a handheld device 102 according to some embodiments of the present disclosure. The system 100 generally includes a device 102 and a console 104 to which the device 102 is to be connected. FIG. 2 is a diagrammatic illustrations of the console 104 coupled to the handheld device 102 illustrating an exemplary embodiment of an end effector 114 for delivering energy to tissue at the one or more target sites of a patient for the treatment of a condition. As illustrated, the device 102 is a handheld device, which includes end effector 114, a shaft 116 operably associated with the end effector 114, and a handle 118 operably associated with the shaft 116. The end effector 114 may be collapsible/retractable and expandable, thereby allowing for the end effector 114 to be minimally invasive (i.e., in a collapsed or retracted state) upon delivery to one or more target sites within a patient and then expanded once positioned at the target site. It should be noted that the terms “end effector” and “therapeutic assembly” may be used interchangeably throughout this disclosure.

For example, a surgeon or other medical professional performing a procedure can utilize the handle 118 to manipulate and advance the shaft 116 to a desired target site, wherein the shaft 116 is configured to locate at least a distal portion thereof intraluminally at a treatment or target site within a portion of the patient associated with tissue to undergo electrotherapeutic stimulation for subsequent treatment of an associated condition or disorder. In the event that the tissue to be treated is a nerve, such that electrotherapeutic stimulation thereof results in treatment of an associated neurological condition, the target site may generally be associated with peripheral nerve fibers. The target site may be a region, volume, or area in which the target nerves are located and may differ in size and shape depending upon the anatomy of the patient. Once positioned, the end effector 114 may be deployed and subsequently deliver energy to the one or more target sites. The energy delivered may be non-therapeutic stimulating energy at a frequency for locating neural tissue and further sensing one or more properties of the neural tissue. For example, the end effector 114 may include an electrode array, which includes at least a subset of electrodes configured to sense the presence of neural tissue at a respective position of each of the electrodes, as well as morphology of the neural tissue, wherein such data may be used for determining, via the console 104, the type of neural tissue, depth of neural tissue, and location of neural tissue.

Based on the identification of the neural tissue type, the console 104 is configured to determine a specific treatment pattern for controlling delivery of energy from the end effector 114 upon the target site at a specific level for a specific period of time to the tissue of interest (i.e., the targeted tissue) sufficient to ensure successful ablation/modulation of the targeted tissue while minimizing and/or preventing collateral damage to surrounding or adjacent non-targeted tissue at the target site. Accordingly, the end effector 114 is able to therapeutically modulate nerves of interest, particularly nerves associated with a peripheral neurological conditional or disorder so as to treat such condition or disorder, while minimizing and/or preventing collateral damage.

For example, the end effector 114 may include at least one energy delivery element, such as an electrode, configured to delivery energy to the target tissue which may be used for sensing presence and/or specific properties of tissue (such tissue including, but not limited to, muscle, nerves, blood vessels, bones, etc.) for therapeutically modulating tissues of interest, such as neural tissue. For example, one or more electrodes may be provided by one or more portions of the end effector 114, wherein the electrodes may be configured to apply electromagnetic neuromodulation energy (e.g., radiofrequency (RF) energy) to target sites. In other embodiments, the end effector 114 may include other energy delivery elements configured to provide therapeutic neuromodulation using various other modalities, such as cryotherapeutic cooling, ultrasound energy (e.g., high intensity focused ultrasound (“HIFU”) energy), microwave energy (e.g., via a microwave antenna), direct heating, high and/or low power laser energy, mechanical vibration, and/or optical power.

In some embodiments, the end effector 114 may include one or more sensors (not shown), such as one or more temperature sensors (e.g., thermocouples, thermistors, etc.), impedance sensors, and/or other sensors. The sensors and/or the electrodes may be connected to one or more wires extending through the shaft 116 and configured to transmit signals to and from the sensors and/or convey energy to the electrodes.

As shown, the device 102 is operatively coupled to the console 104 via a wired connection, such as cable 120. It should be noted, however, that the device 102 and console 104 may be operatively coupled to one another via a wireless connection. The console 104 is configured to provide various functions for the device 102, which may include, but is not limited to, controlling, monitoring, supplying, and/or otherwise supporting operation of the device 102. For example, when the device 102 is configured for electrode-based, heat-element-based, and/or transducer-based treatment, the console 104 may include an energy generator 106 configured to generate RF energy (e.g., monopolar, bipolar, or multi-polar RF energy), pulsed electrical energy, microwave energy, optical energy, ultrasound energy (e.g., intraluminally-delivered ultrasound and/or HIFU), direct heat energy, radiation (e.g., infrared, visible, and/or gamma radiation), and/or another suitable type of energy.

In some embodiments, the console 104 may include a controller 107 communicatively coupled to the device 102. However, in the embodiments described herein, the controller 107 may generally be carried by and provided within the handle 118 of the device 102. The controller 107 is configured to initiate, terminate, and/or adjust operation of one or more electrodes provided by the end effector 114 directly and/or via the console 104. For example, the controller 107 can be configured to execute an automated control algorithm and/or to receive control instructions from an operator (e.g., surgeon or other medical professional or clinician). For example, the controller 107 and/or other components of the console 104 (e.g., processors, memory, etc.) can include a computer-readable medium carrying instructions, which when executed by the controller 107, causes the device 102 to perform certain functions (e.g., apply energy in a specific manner, detect impedance, detect temperature, detect nerve locations or anatomical structures, etc.). A memory includes one or more of various hardware devices for volatile and non-volatile storage, and can include both read-only and writable memory. For example, a memory can comprise random access memory (RAM), CPU registers, read-only memory (ROM), and writable non-volatile memory, such as flash memory, hard drives, floppy disks, CDs, DVDs, magnetic storage devices, tape drives, device buffers, and so forth. A memory is not a propagating signal divorced from underlying hardware; a memory is thus non-transitory.

The console 104 may further be configured to provide feedback to an operator before, during, and/or after a treatment procedure via evaluation/feedback algorithms 110. For example, the evaluation/feedback algorithms 110 can be configured to provide information associated with the location of nerves at the treatment site, the temperature of the tissue at the treatment site, and/or the effect of the therapeutic neuromodulation on the nerves at the treatment site. In certain embodiments, the evaluation/feedback algorithm 110 can include features to confirm efficacy of the treatment and/or enhance the desired performance of the system 100. For example, the evaluation/feedback algorithm 110, in conjunction with the controller 107, can be configured to monitor temperature at the treatment site during therapy and automatically shut off the energy delivery when the temperature reaches a predetermined maximum (e.g., when applying RF energy) or predetermined minimum (e.g., when applying cryotherapy). In other embodiments, the evaluation/feedback algorithm 110, in conjunction with the controller 107, can be configured to automatically terminate treatment after a predetermined maximum time, a predetermined maximum impedance rise of the targeted tissue (i.e., in comparison to a baseline impedance measurement), a predetermined maximum impedance of the targeted tissue, and/or other threshold values for biomarkers associated with autonomic function. This and other information associated with the operation of the system 100 can be communicated to the operator via a graphical user interface (GUI) 112 provided via a display on the console 104 and/or a separate display (not shown) communicatively coupled to the console 104, such as a tablet or monitor, to thereby provide visual and/or audible alerts to the operator. The GUI 112 may generally provide operational instructions for the procedure, such as directing the operator to select which sino-nasal cavity to treat, indicating when the device 102 is primed and ready to perform treatment, and further providing status of therapy during the procedure, including indicating when the treatment is complete.

For example, in some embodiments, the end effector 114 and/or other portions of the system 100 can be configured to detect various parameters of the heterogeneous tissue at the target site to determine the anatomy at the target site (e.g., tissue types, tissue locations, vasculature, bone structures, foramen, sinuses, etc.), locate nerves and/or other structures, and allow for neural mapping. For example, the end effector 114 may be configured to detect impedance, dielectric properties, temperature, and/or other properties that indicate the presence of neural fibers in the target region.

As shown in FIG. 1A, the console 104 further includes a monitoring system 108 configured to receive data from the end effector 114 (i.e., detected electrical and/or thermal measurements of tissue at the target site), specifically sensed by appropriate sensors (e.g., temperature sensors and/or impedance sensors, or the like), and process this information to identify the presence of nerves, the location of nerves, neural activity at the target site, and/or other properties of the neural tissue, such a physiological properties (e.g., depth), bioelectric properties, and thermal properties. The nerve monitoring system 108 can be operably coupled to the electrodes and/or other features of the end effector 114 via signal wires (e.g., copper wires) that extend through the cable 120 and through the length of the shaft 116. In other embodiments, the end effector 114 can be communicatively coupled to the nerve monitoring system 108 using other suitable communication means.

The nerve monitoring system 108 can determine neural locations and activity before therapeutic neuromodulation to determine precise treatment regions corresponding to the positions of the desired nerves. The nerve monitoring system 108 can further be used during treatment to determine the effect of the therapeutic neuromodulation, and/or after treatment to evaluate whether the therapeutic neuromodulation treated the target nerves to a desired degree. This information can be used to make various determinations related to the nerves proximate to the target site, such as whether the target site is suitable for neuromodulation. In addition, the nerve monitoring system 108 can also compare the detected neural locations and/or activity before and after therapeutic neuromodulation, and compare the change in neural activity to a predetermined threshold to assess whether the application of therapeutic neuromodulation was effective across the treatment site. For example, the nerve monitoring system 108 can further determine electroneurogram (ENG) signals based on recordings of electrical activity of neurons taken by the end effector 114 before and after therapeutic neuromodulation. Statistically meaningful (e.g., measurable or noticeable) decreases in the ENG signal(s) taken after neuromodulation can serve as an indicator that the nerves were sufficiently ablated. Additional features and functions of the nerve monitoring system 108, as well as other functions of the various components of the console 104, including the evaluation/feedback algorithms 110 for providing real-time feedback capabilities for ensuring optimal therapy for a given treatment is administered, are described in at least U.S. Publication No. 2016/0331459 and U.S. Publication No. 2018/0133460, the contents of each of which are incorporated by reference herein in their entireties.

The device 102 provides access to target sites associated with peripheral nerves for the subsequent neuromodulation of such nerves and treatment of a corresponding peripheral neurological condition or disorder. Accordingly, the devices, systems, and methods of the present invention are useful in detecting, identifying, and precision targeting nerves associated with the peripheral nervous system for treatment of corresponding peripheral neurological conditions or disorders. In the present case, the condition or disorder is headache disorders.

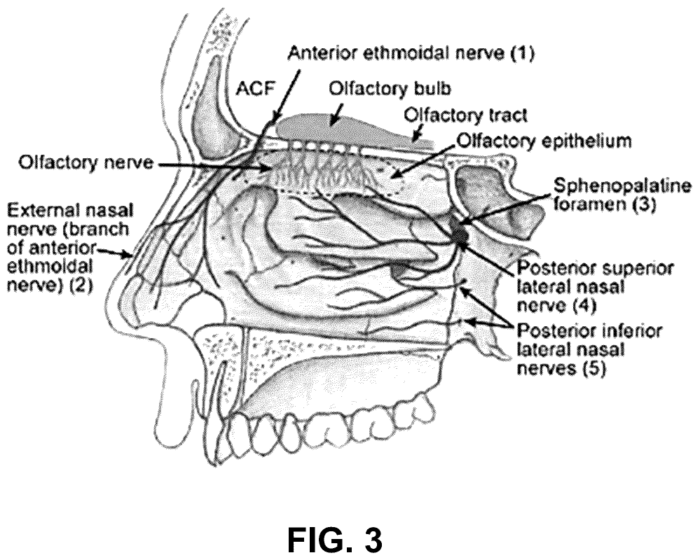

FIG. 3 is a cut-away side view illustrating the anatomy of a lateral sino-nasal wall. The sphenopalatine foramen (SPF) is an opening or conduit defined by the palatine bone and the sphenoid bone through which the sphenopalatine vessels and the posterior superior sino-nasal nerves travel into the sino-nasal cavity. More specifically, the orbital and sphenoidal processes of the perpendicular plate of the palatine bone define the sphenopalatine notch, which is converted into the SPF by the articulation with the surface of the body of the sphenoid bone.

The location of the SPF is highly variable within the posterior region of the lateral sino-nasal cavity, which makes it difficult to visually locate the SPF. Typically, the SPF is located in the middle meatus. However, anatomical variations also result in the SPF being located in the superior meatus or at the transition of the superior and middle meatuses. In certain individuals, for example, the inferior border of the SPF has been measured at about 19 mm above the horizontal plate of the palatine bone (i.e., the sino-nasal sill), which is about 13 mm above the horizontal lamina of the inferior turbinate and the average distance from the sino-nasal sill to the SPF is about 64.4 mm, resulting in an angle of approach from the sino-nasal sill to the SPF of about 11.4°. However, studies to measure the precise location of the SPF are of limited practical application due to the wide variation of its location.

The anatomical variations of the SPF are expected to correspond to alterations of the autonomic and vascular pathways traversing into the sino-nasal cavity. In general, it is thought that the posterior sino-nasal nerves (also referred to as lateral posterior superior sino-nasal nerves) branch from the pterygopalatine ganglion (PPG), which is also referred to as the sphenopalatine ganglion (SPG), through the SPF to enter the lateral sino-nasal wall of the sino-nasal cavity, and the sphenopalatine artery passes from the pterygopalatine fossa through the SPF on the lateral sino-nasal wall. The sphenopalatine artery branches into two main portions: the posterior lateral sino-nasal branch and the posterior septal branch. The main branch of the posterior lateral sino-nasal artery travels inferiorly into the inferior turbinate (e.g., between about 1.0 mm and 1.5 mm from the posterior tip of the inferior turbinate), while another branch enters the middle turbinate MT and branches anteriorly and posteriorly.

Beyond the SPF, studies have shown that over 30% of human patients have one or more accessory foramen that also carries arteries and nerves into the sino-nasal cavity. The accessory foramen are typically smaller than the SPF and positioned inferior to the SPF. For example, there can be one, two, three or more branches of the posterior sino-nasal artery and posterior sino-nasal nerves that extend through corresponding accessory foramen. The variability in location, size, and quantity associated with the accessory foramen and the associated branching arteries and nerves that travel through the accessory foramen gives rise to a great deal of uncertainty regarding the positions of the vasculature and nerves of the sphenopalatine region. Furthermore, the natural anatomy extending from the SPF often includes deep inferior and/or superior grooves that carry neural and arterial pathways, which make it difficult to locate arterial and neural branches. For example the grooves can extend more than 5 mm long, more than 2 mm wide, and more than 1 mm deep, thereby creating a path significant enough to carry both arteries and nerves. The variations caused by the grooves and the accessory foramen in the sphenopalatine region make locating and accessing the arteries and nerves (positioned posterior to the arteries) extremely difficult for surgeons.

Recent microanatomic dissection of the pterygopalatine fossa have further evidenced the highly variable anatomy of the region surrounding the SPF, showing that a multiplicity of efferent rami that project from the SPG to innervate the orbit and sino-nasal mucosa via numerous groups of small nerve fascicles, rather than an individual postganglionic autonomic nerves (e.g., the posterior sino-nasal nerve). Studies have shown that at least 87% of humans have microforamina and micro rami in the palatine bone.

As previously described herein, each of the anterior ethmoidal nerve (AEN), posterior nasal nerve (PNN), and sphenopalatine ganglion (SPG) region play a role in headache disorders. The SPG has been implicated in many headache disorders. SPG stimulation has already shown to reduce headache and migraine symptom. Side effects of SPG treatment include sensitivity in the face, gums and teeth. As a result alternative target regions maybe required.

The PNN is an accessible trigeminal branch of the SPG that innervates the nasal cavity along the turbinates and along the lateral wall. The PNN consists of trigeminal afferent branches and postganglionic parasympathetic fibers which release neuropeptides such as calcitonin gene-related peptide (CGRP), substance P and vasoactive intestinal peptide (e.g., widens the blood vessels) upon stimulus and irritation. These neuropeptides, upon activation, can contribute significantly to pain and sensation and may contribute to symptoms, such as headache and facial pain. Headaches/migraines usually accompany nasal symptoms, whether the symptoms occur due to migraine or vice versa is controversial. Despite this, it is believed that treatment of the pathologies of the nose will reduce frequency and intensity of headaches.

The anterior ethmoidal branch is a branch of the ophthalmic nerve (trigeminal nerve) and is a continuation of the nasociliary nerve. The AEN consists of internal and external branches. The internal branches of the AEN travel along the mucous membrane on the front part of the septum and the external branches travel through the ethmoidal groove of the nasal bone to the tip of the nose. The AEN has also been implicated in headaches due to activation/stimulation of nociceptive non-myelinated C fibers. Lateral branches of the AEN are easily accessible along the nasal septum. Studies have shown that simply blocking the external branch using injectable anesthetic can relieve some of the headache symptoms. The use of stimulation to identify the sensory nerve (50-300 Hz, 500 mA and 0.7V) and ablating the AEN with a low profile multipoint ablation system could potentially extend the therapy duration experienced by anesthetic blocks.

The secretion of neuropeptides CGRP and substance P from the trigeminal nerve and VIP from parasympathetic fibers are involved in the pathophysiology of CRS. The neuropeptides are released upon stimulus/irritation of trigeminal afferent branches and parasympathetic fibers. Ethmoidal sinuses receive sensory and parasympathetic information from the trigeminal branches, including anterior ethmoid and posterior ethmoid nerves (from the nasociliary nerve), and posterior lateral superior nasal nerve (from the pterygopalatine ganglion). Ablating these trigeminal branches could relieve some of the sino-nasal symptoms experienced with ethmoidal sinusitis. Furthermore, the posterior ethmoidal nerve is often an inaccessible nerve due either being absent in the patient or being surrounded by important olfactory nerves (responsible for sense of smell). Ablating accessible trigeminal branches, such as the lateral superior posterior nasal branches (including along the middle meatus) and AEN, may relieve some of these symptoms.

Accordingly, embodiments of the present disclosure are configured to therapeutically modulate targeted neural tissue at precise and focused treatment sites corresponding to at least one of AEN tissue, PNN tissue, and SPG nerve cells for the treatment of headache disorders.

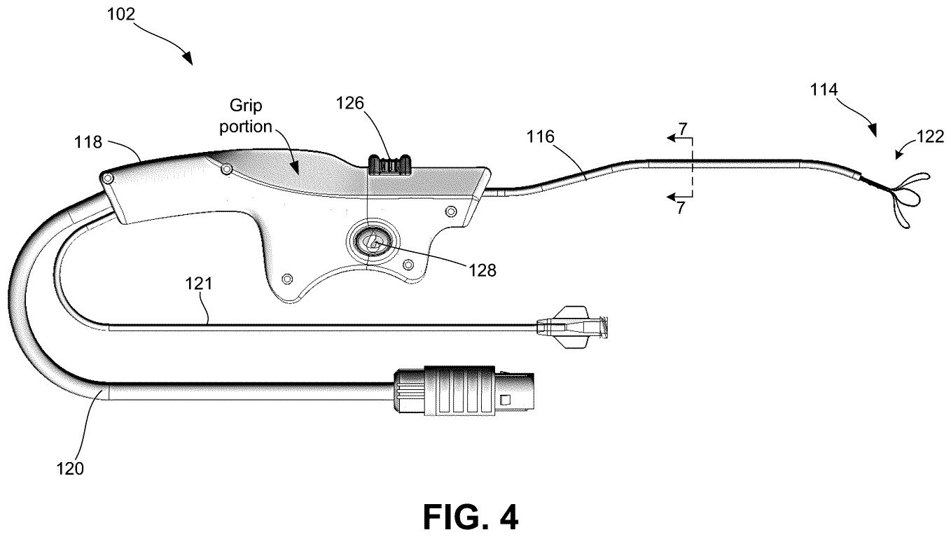

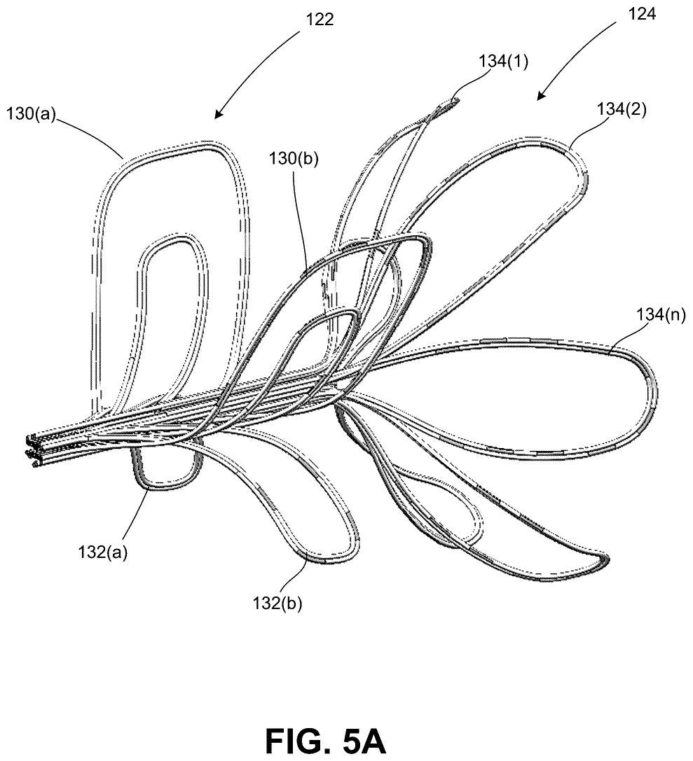

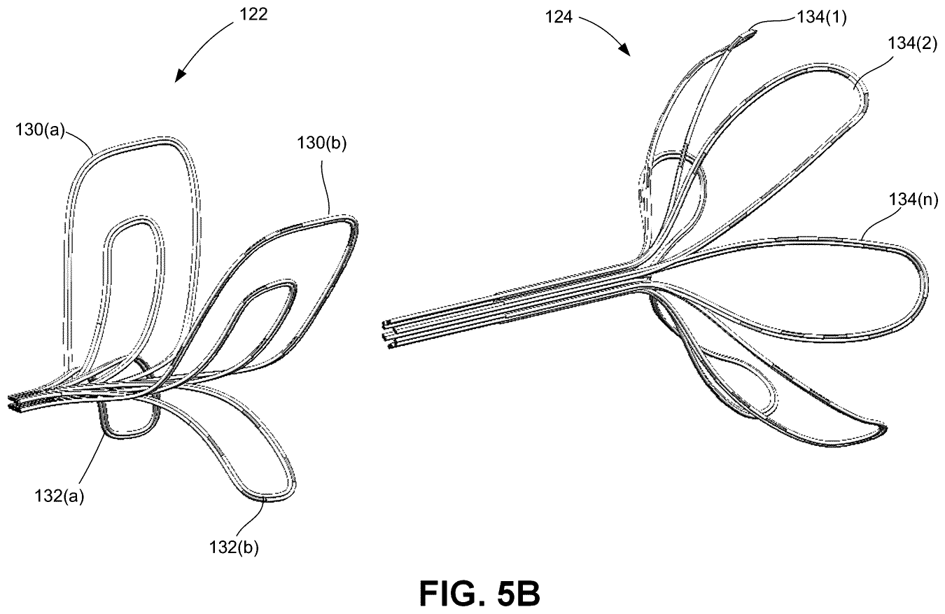

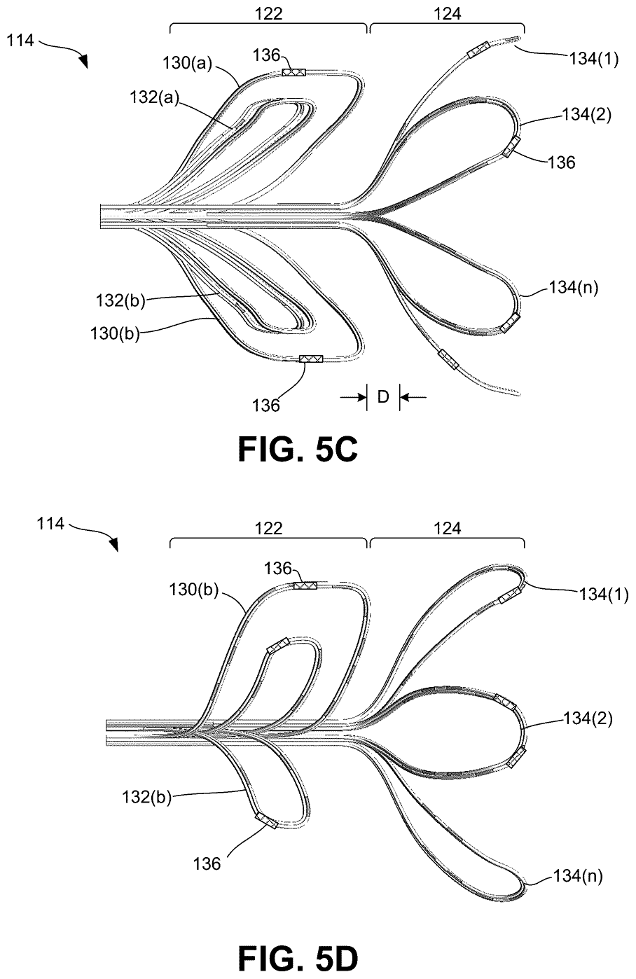

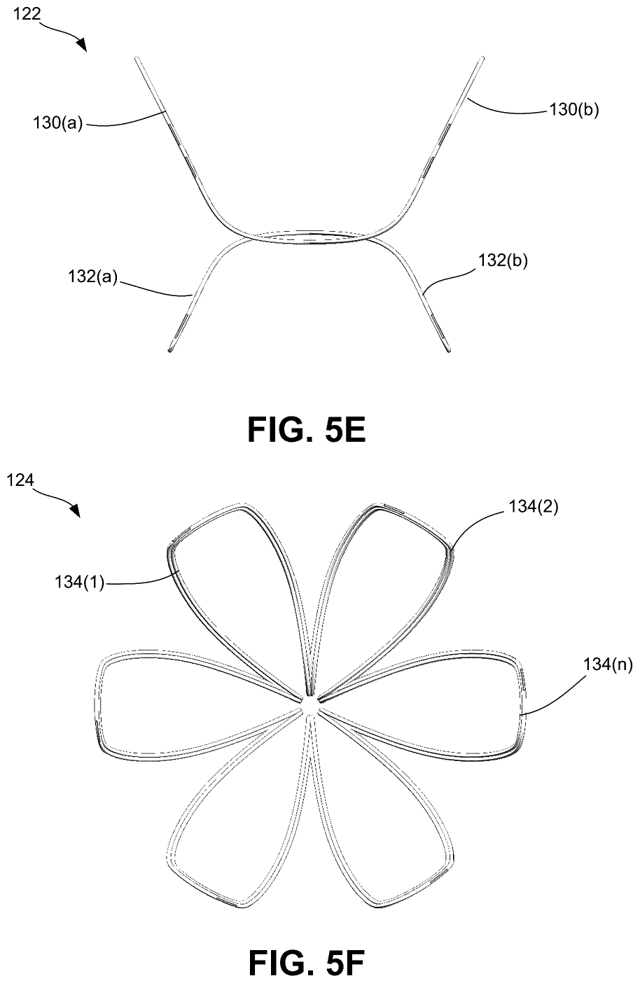

FIG. 4 is a side view of one embodiment of a handheld device for providing therapeutic neuromodulation consistent with the present disclosure. As illustrated, the device 102 includes an end effector 114 transformable between a retracted configuration and an expanded deployed configuration, a shaft 116 operably associated with the end effector 114, and a handle 118 operably associated with the shaft 116. The end effector 114 includes at least a first segment 122. In some embodiments, the end effector is a multi-segmented end effector, and thus includes two or more segments. For example, as described herein, the end effector may include a second segment 124 spaced apart from the first segment 122. The first segment 122 is generally positioned closer to a distal end of the shaft 116, and is thus sometimes referred to herein as the proximal segment 122, while the second segment 124 is generally positioned further from the distal end of the shaft 116 and is thus sometimes referred to herein as the distal segment 124. Each of the first and second segments 122 and 124 is transformable between a retracted configuration, which includes a low-profile delivery state to facilitate intraluminal delivery of the end effector 114 to a treatment site within the sino-nasal region, and a deployed configuration, which includes an expanded state, as shown in FIG. 4 and further illustrated in FIGS. 5A-5F.

The handle 118 includes at least a first mechanism 126 for deployment of the multi-segment end effector 114, notably the first and second segments 122, 124, from the retracted configuration to the deployed configuration and a second mechanism 128, separate from the first mechanism 124, for control of energy output by either of the first and second segments 122, 124 of the end effector 114, specifically electrodes or other energy elements provided by first and/or second segments 122, 124. The handheld device 102 may further include an auxiliary line 121, which may provide a fluid connection between a fluid source, for example, and the shaft 116 such that fluid may be provided to a target site via the distal end of the shaft 116. In some embodiments, the auxiliary line 121 may provide a connection between a vacuum source and the shaft 116, such that the device 102 may include suction capabilities (via the distal end of the shaft 116).

FIGS. 5A, 5B, 5C, 5D, 5E, and 5F are enlarged views of the multi-segment end effector 114, illustrating various views of the first and second segments 122, 124 in greater detail. FIG. 5A is an enlarged, perspective view of the multi-segment end effector 114. FIG. 5B is an exploded, perspective view of the multi-segment end effector 114. FIGS. 5C and 5D are enlarged, top and side views, respectively, of the multi-segment end effector 114. FIG. 5E is an enlarged, front (proximal facing) view of the first segment 122 of the multi-segment end effector 114. FIG. 5F is an enlarged, front (proximal facing) view of the second segment 124 of the multi-segment end effector 114.

As illustrated, the first segment 122 includes at least a first set of flexible support elements, generally in the form of wires, arranged in a first configuration, and the second segment 124 includes a second set of flexible support elements, also in the form of wires, arranged in a second configuration. The first and second sets of flexible support elements include composite wires having conductive and elastic properties. For example, in some embodiments, the composite wires include a shape memory material, such as nitinol. The flexible support elements may further include a highly lubricious coating, which may allow for desirable electrical insulation properties as well as desirable low friction surface finish. Each of the first and second segments 122, 124 is transformable between a retracted configuration and an expanded deployed configuration such that the first and second sets of flexible support elements are configured to position one or more electrodes provided on the respective segments (see electrodes 136 in FIGS. 5E and 5F) into contact with one or more target sites when in the deployed configuration.

As shown, when in the expanded deployed configuration, the first set of support elements of the first segment 122 includes at least a first pair of struts 130a, 130b, each comprising a loop (or leaflet) shape and extending in an upward direction and a second pair of struts 132a, 132b, each comprising a loop (or leaflet) shape and extending in a downward direction, generally in an opposite direction relative to at least the first pair of struts 130a, 130b. It should be noted that the terms upward and downward are used to describe the orientation of the first and second segments 122, 124 relative to one another. More specifically, the first pair of struts 130a, 130b generally extend in an outward inclination in a first direction relative to a longitudinal axis of the multi-segment end effector 114 and are spaced apart from one another. Similarly, the second pair of struts 132a, 132b extend in an outward inclination in a second direction substantially opposite the first direction relative to the longitudinal axis of the multi-segment end effector and spaced apart from one another.

The second set of support elements of the second segment 124, when in the expanded deployed configuration, includes a second set of struts 134(1), 134(2), 134(n) (approximately six struts), each comprising a loop shape extending outward to form an open-ended circumferential shape. As shown, the open-ended circumferential shape generally resembles a blooming flower, wherein each looped strut 134 may generally resemble a flower petal. It should be noted that the second set of struts 134 may include any number of individual struts and is not limited to six, as illustrated. For example, in some embodiments, the second segment 124 may include two, three, four, five, six, seven, eight, nine, ten, or more struts 134.

The first and second segments 122, 124, specifically struts 130, 132, and 134 include one or more energy delivery elements, such as a plurality of electrodes 136. It should be noted that any individual strut may include any number of electrodes 136 and is not limited to one electrode, as shown. In the expanded state, the struts 130, 132, and 134 can position any number of electrodes 136 against tissue at a target site within the sino-nasal region. The electrodes 136 can apply bipolar or multi-polar radiofrequency (RF) energy to the target site to therapeutically modulate targeted tissue. In various embodiments, the electrodes 136 can be configured to apply pulsed RF energy with a desired duty cycle (e.g., 1 second on/0.5 seconds off) to regulate the temperature increase in the target tissue.

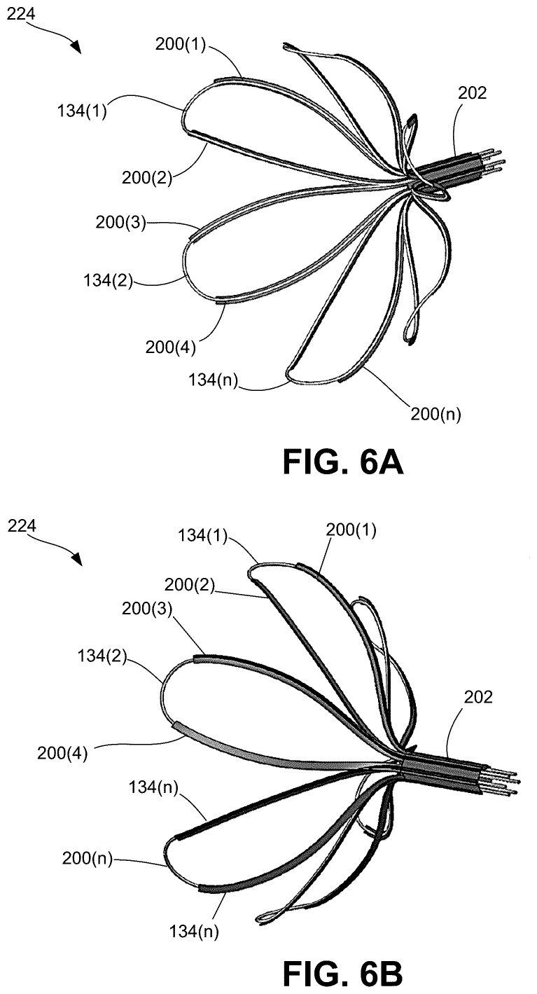

The first and second segments 122, 124 and the associated struts 130, 132, and 134 can have sufficient rigidity to support the electrodes 136 and position or press the electrodes 136 against tissue at the target site. In addition, each of the expanded first and second segments 122, 124 can press against surrounding anatomical structures proximate to the target site (e.g., the turbinates, the palatine bone, etc.) and the individual struts 130, 132, 134 can at least partially conform to the shape of the adjacent anatomical structures to anchor the end effector 114 In addition, the expansion and conformability of the struts 130, 132, 134 can facilitate placing the electrodes 136 in contact with the surrounding tissue at the target site. The electrodes 136 can be made from platinum, iridium, gold, silver, stainless steel, platinum-iridium, cobalt chromium, iridium oxide, polyethylenedioxythiophene (PEDOT), titanium, titanium nitride, carbon, carbon nanotubes, platinum grey, Drawn Filled Tubing (DFT) with a silver core, and/or other suitable materials for delivery RF energy to target tissue. In some embodiments, such as illustrated in FIG. 6, a strut may include an outer jacket surrounding a conductive wire, wherein portions of the outer jacket are selectively absent along a length of the strut, thereby exposing the underlying conductive wire so as to act as an energy delivering element (i.e., an electrode) and/or sensing element, as described in greater detail herein.

In certain embodiments, each electrode 136 can be operated independently of the other electrodes 136. For example, each electrode can be individually activated and the polarity and amplitude of each electrode can be selected by an operator or a control algorithm (e.g., executed by the controller 107 previously described herein. The selective independent control of the electrodes 136 allows the end effector 114 to deliver RF energy to highly customized regions. For example, a select portion of the electrodes 136 can be activated to target neural fibers in a specific region while the other electrodes 136 remain inactive. In certain embodiments, for example, electrodes 136 may be activated across the portion of the second segment 124 that is adjacent to tissue at the target site, and the electrodes 136 that are not proximate to the target tissue can remain inactive to avoid applying energy to non-target tissue. Such configurations facilitate selective therapeutic modulation of nerves on the lateral sino-nasal wall within one nostril without applying energy to structures in other portions of the sino-nasal cavity.

The electrodes 136 are electrically coupled to an RF generator (e.g., the generator 106 of FIG. 1A) via wires (not shown) that extend from the electrodes 136, through the shaft 116, and to the RF generator. When each of the electrodes 136 is independently controlled, each electrode 136 couples to a corresponding wire that extends through the shaft 116. In other embodiments, multiple electrodes 116 can be controlled together and, therefore, multiple electrodes 116 can be electrically coupled to the same wire extending through the shaft 116. As previously described, the RF generator and/or components operably coupled (e.g., a control module) thereto can include custom algorithms to control the activation of the electrodes 136. For example, the RF generator can deliver RF power at about 460-480 kHz (+ or −5 kHz) to the electrodes 136, and do so while activating the electrodes 136 in a predetermined pattern selected based on the position of the end effector 114 relative to the treatment site and/or the identified locations of the target nerves. The RF generator is able to provide bipolar low power (10 watts with maximum setting of 50 watts) RF energy delivery, and further provide multiplexing capabilities (across a maximum of 30 channels).

Once deployed, the first and second segments 122, 124 contact and conform to a shape of the respective locations, including conforming to and complementing shapes of one or more anatomical structures at the respective locations. In turn, the first and second segments 122, 124 become accurately positioned within the sino-nasal cavity to subsequently deliver, via one or more electrodes 136, precise and focused application of RF thermal energy to the one or more target sites to thereby therapeutically modulate associated neural structures.

For example, the first set of flexible support elements of the first segment 122 conforms to and complements a shape of a first anatomical structure at the first location when the first segment 122 is in the deployed configuration and the second set of flexible support elements of the second segment 124 conforms to and complements a shape of a second anatomical structure at the second location when the second segment is in the deployed configuration. The first and second anatomical structures may include, but are not limited to, inferior turbinate, middle turbinate, superior turbinate, inferior meatus, middle meatus, superior meatus, pterygopalatine region, pterygopalatine fossa, sphenopalatine foramen, accessory sphenopalatine foramen(ae), and sphenopalatine micro-foramen(ae).

As illustrated in FIG. 5E, the first segment 122 comprises a bilateral geometry. In particular, the first segment 122 includes two identical sides, including a first side formed of struts 130a, 132a and a second side formed of struts 130b, 132b. This bilateral geometry allows at least one of the two sides to conform to and accommodate an anatomical structure within the sino-nasal cavity when the first segment 122 is in an expanded state. For example, when in the expanded state, the plurality of struts 130a, 132a contact multiple locations along multiple portions of the anatomical structure and electrodes provided by the struts are configured to emit energy at a level sufficient to create multiple micro-lesions in tissue of the anatomical structure that interrupt neural signals to mucus producing and/or mucosal engorgement elements. In particular, struts 130a, 132a conform to and complement a shape of a lateral attachment and posterior-inferior edge of the middle turbinate when the first segment 122 is in the deployed configuration, thereby allowing for both sides of the anatomical structure to receive energy from the electrodes. By having this independence between first and second side (i.e., right and left side) configurations, the first segment 122 is a true bilateral device. By providing a bilateral geometry, the multi-segment end effector 114 does not require a repeat use configuration to treat the other side of the anatomical structure, as both sides of the structure are accounted at the same time due to the bilateral geometry. The resultant micro-lesion pattern can be repeatable and is predictable in both macro element (depth, volume, shape parameter, surface area) and can be controlled to establish low to high effects of each, as well as micro elements (the thresholding of effects within the range of the macro envelope can be controlled).

It should be noted that, in some embodiments, a given retractable and expandable segment of the end effector may include one or more flexible printed circuit board (PCB) members provided thereon. The flexible PCB members are composed of a flexible material capable of moving (e.g., bending, twisting, folding, etc.) between various positions in correspondence with movement of the underlying retractable and expandable segment to which it is attached. Each flexible PCB member further includes one or more energy delivering elements (e.g., electrodes) provided thereon and configured to deliver energy to tissue associated with one or more target sites in the sino-nasal cavity.

FIGS. 6A and 6B are enlarged perspective views of the second (distal) segment 124 of the multi-segment end effector 114 including a flexible printed circuit board (PCB) assembly operably associated therewith.

As shown, each of the struts or support elements 134(1)-134(6) of the second (distal) segment 134 includes loop-like or leaflet-like shape. Accordingly, the struts or support structures 134 may also be referred to herein as leaflets. Each leaflet 134 includes a pair of flexible PCB members 200 affixed to a portion thereof. For example, a first leaflet 134(1) includes a set of flexible PCB members 200(1) and 200(2) coupled thereto, a second leaflet 134(2) includes a second set of flexible PCB members 200(3) and 200(4) coupled thereto, and so on. Accordingly, in the present embodiment, the flexible PCB assembly includes twelve individual flexible PCB members 200, wherein each of the six leaflets 134 of the distal segment 124 includes a pair of flexible PCB members 200 attached thereto.

Each of the flexible PCB members 200 includes a PCB substrate or base layer composed of a flexible material upon which one or more electronic components are provided, such as, for example, energy delivering elements (i.e., electrodes) and/or sensors. As shown, as a result of a flexible substrate material, each of the flexible PCB members 200 is capable of moving (e.g., bending, twisting, folding, etc.) between various positions in correspondence with movement of the underlying retractable and expandable leaflet 134 to which it is attached. As will be described in greater detail herein, each flexible PCB member 200 further includes one or more energy delivering elements (e.g., electrodes) provided thereon and configured to deliver energy to tissue associated with one or more target sites in the sino-nasal cavity. In this manner, upon deployment of the second (distal) segment 124 to the expanded configuration, each of the plurality of flexible PCB members 200 attached to the distal segment 124 is able to correspondingly move and transition into the specific geometry of leaflets 134, such that, once deployed, the flexible PCB members 200 contact and conform to a shape of the respective location, including conforming to and complementing shapes of one or more anatomical structures at the respective locations. In turn, the plurality of flexible PCB members 200 become accurately positioned within the sino-nasal cavity to subsequently deliver, via one or more electrodes, precise and focused application of energy to targeted tissue at the one or more target sites. Additional features and functions of the flexible PCB members, including methods of manufacture and other details, are described in at least international PCT Publication No. WO/2022/043757, the content of which is incorporated by reference herein in its entirety.

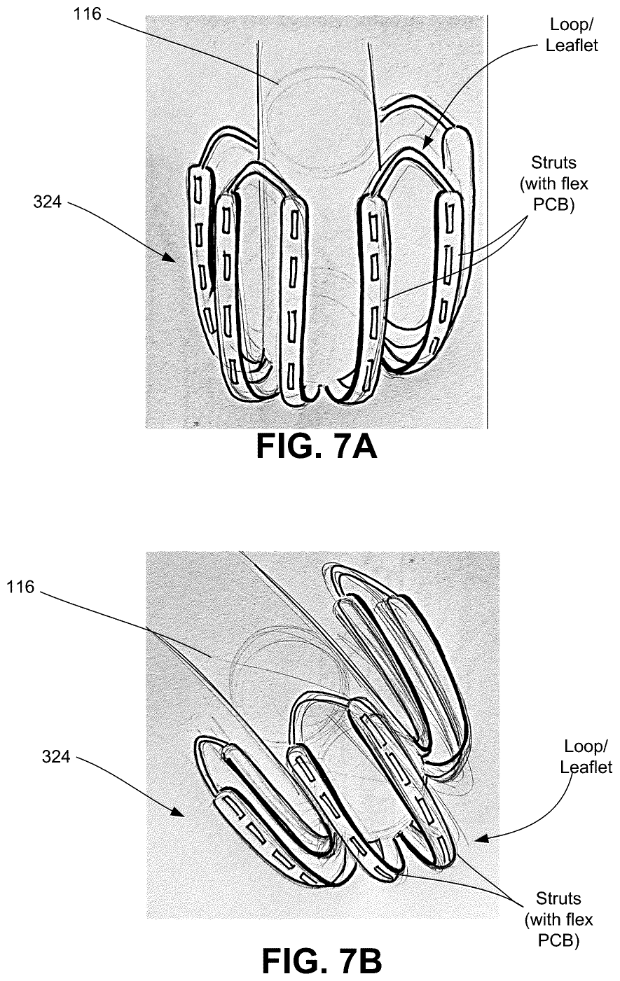

FIGS. 7A and 7B are enlarged perspective views of another embodiment of an end effector consistent with the present disclosure. As shown, the end effector may include a segment 324 comprising a plurality of loop-shaped struts or leaflets that are generally extending back towards the shaft of the device when in an expanded, deployed state. Each loop-shaped strut or leaflet may be equidistantly spaced apart from one another, to thereby improve consistency of lesion depth upon delivery of energy therefrom.

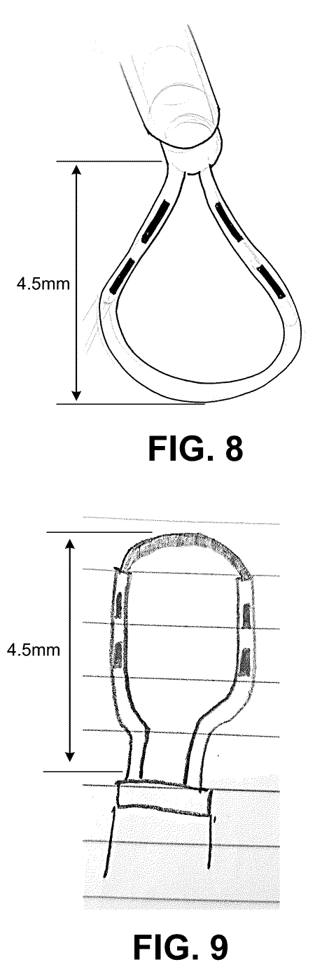

FIGS. 8 and 9 are side views showing single loop-shaped struts of an end effector consistent with the present disclosure that can be used for delivering energy to target tissue at target sites within the sino-nasal cavity.

The device of the present disclosure can be used in treating headache disorders by therapeutically modulating neural structures associated with headache disorders.

For example, the end effector may be used for delivering energy to target sites within the sino-nasal cavity of a patient that include, but are not limited to, anterior ethmoidal nerve (AEN) tissue, posterior nasal nerve (PNN) tissue, and sphenopalatine ganglion (SPG) nerve cells. For example, the energy delivered to target site(s) within the sino-nasal cavity is sufficient to therapeutically treat neural tissues of interest associated with the AEN tissue, PNN tissue, and/or SPG nerve cells. In particular, energy delivered to the target site(s) may be sufficient to therapeutically modulate or interrupt neural signals associated with AEN tissue, PNN tissue, and/or SPG nerve cells, thereby reducing the frequency and intensity of headache disorder-related systems, thereby treating headache disorders at their core.

Once the end effector is delivered within the sino-nasal cavity and positioned relative to the desired target site(s) (i.e., relative to the AEN, PNN, and/or SPG), portions of the end effector (i.e., flexible support elements) can transition to a deployed state, having a specific shape and/or size corresponding to anatomical structures within the sino-nasal cavity and associated with target sites to undergo delivery of therapeutic energy for treatment of headache disorders. As such, once deployed, the energy delivery element(s) can be positioned at desired locations for focused application of energy to the underlying targeted neural tissue at the one or more target sites.

In the instant embodiment, the end effector of the present invention can be used to deliver treatment energy to one or more tissues at one or more target sites within a sino-nasal cavity of the patent to thereby cause multipoint interruption and/or multipoint modulation of one or more targeted neural tissues for the treatment of a headache disorder.

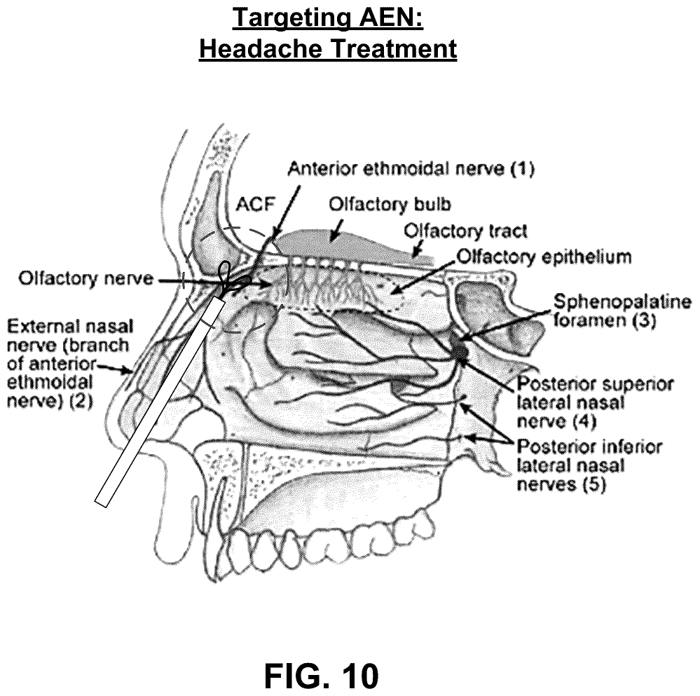

For example, FIG. 10 is a cut-away side view illustrating one approach for delivering an end effector to a target site within a sino-nasal region of a patient and subsequently delivering energy to one or more target tissues associated with AEN tissue for the treatment of headache disorder in accordance with embodiments of the present disclosure.

The anterior ethmoidal branch is a branch of the ophthalmic nerve (trigeminal nerve) and is a continuation of the nasociliary nerve. The AEN consists of internal and external branches. The internal branches of the AEN travel along the mucous membrane on the front part of the septum and the external branches travel through the ethmoidal groove of the nasal bone to the tip of the nose. The AEN has also been implicated in headaches due to activation/stimulation of nociceptive non-myelinated C fibers. Lateral branches of the AEN are easily accessible along the nasal septum. Studies have shown that simply blocking the external branch using injectable anesthetic can relieve some of the headache symptoms. The use of stimulation to identify the sensory nerve (50-300 Hz, 500 mA and 0.7V) and ablating the AEN with a low profile multipoint ablation system could potentially extend the therapy duration experienced by anesthetic blocks.

As such, the delivery of treatment energy from the end effector can be used to ablate/stimulate the AEN region (or in combination with ablation of PNN) through multipoint interruption (ablation) or multipoint modulation (stimulation) of nocicepetive c fibers could reduce the frequency and intensity of headache disorders.

More specifically, the delivery of treatment energy can effectively create targeted micro-lesions in targeted neural tissue (including AEN tissue and/or PNN tissue) to thereby interrupt associated neural signals from passing therethrough.

The secretion of neuropeptides CGRP and substance P from the trigeminal nerve and VIP from parasympathetic fibers are involved in the pathophysiology of CRS. The neuropeptides are released upon stimulus/irritation of trigeminal afferent branches and parasympathetic fibers. Ethmoidal sinuses receive sensory and parasympathetic information from the trigeminal branches, including anterior ethmoid and posterior ethmoid nerves (from the nasociliary nerve), and posterior lateral superior nasal nerve (from the pterygopalatine ganglion). Ablating these trigeminal branches could relieve some of the sino-nasal symptoms experienced with ethmoidal sinusitis. Furthermore, the posterior ethmoidal nerve is often an inaccessible nerve due either being absent in the patient or being surrounded by important olfactory nerves (responsible for sense of smell). Ablating accessible trigeminal branches, such as the lateral superior posterior nasal branches (including along the middle meatus) and AEN, may relieve some of these symptoms. Furthermore, the present invention is able to deliver precise levels of treatment energy such that energy is sufficient to create the targeted micro-lesions in the AEN tissue without damaging olfactory nerves to thereby preserve olfactory function.

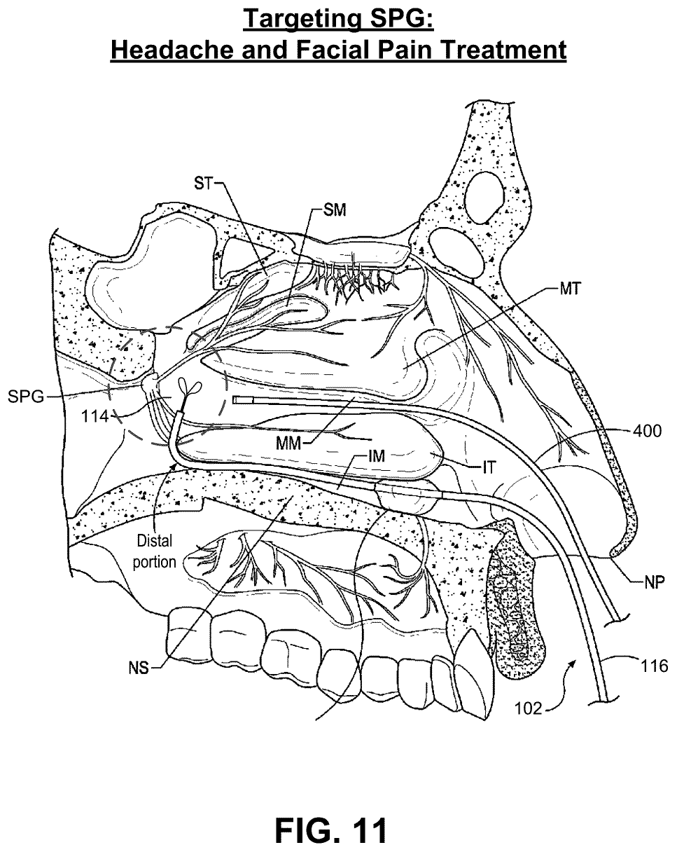

FIG. 11 is a cut-away side view illustrating another approach for delivering an end effector to a target site within a sino-nasal region of a patient and subsequently delivering energy to one or more target tissues associated with SPG nerve cells for the treatment of headache disorder in accordance with embodiments of the present disclosure.

As shown, the distal portion of the shaft 116 extends into the nasal passage NP, through the inferior meatus IM between the inferior turbinate IT and the nasal sill NS, and around the posterior portion of the inferior turbinate IT where the end effector 114 is deployed at a treatment site. The treatment site can be located proximate to the SPG region.

In various embodiments, the distal portion of the shaft 116 may be guided into position at the target site via a guidewire (not shown) using an over-the-wire (OTW) or a rapid exchange (RX) technique. For example, the end effector 114 can include a channel for engaging the guidewire. Intraluminal delivery of the end effector 114 can include inserting the guide wire into an orifice in communication with the nasal cavity (e.g., the nasal passage or mouth), and moving the shaft 116 and/or the end effector 114 along the guide wire until the end effector 114 reaches a target site (e.g., proximate the SPG region).

Yet still, in further embodiments, the neuromodulation device 102 can be configured for delivery via a guide catheter or introducer sheath (not shown) with or without using a guide wire. The introducer sheath can first be inserted intraluminally to the target site in the nasal region, and the distal portion of the shaft 116 can then be inserted through the introducer sheath. At the target site, the end effector 114 can be deployed through a distal end opening of the introducer sheath or a side port of the introducer sheath. In certain embodiments, the introducer sheath can include a straight portion and a pre-shaped portion with a fixed curve (e.g., a 5 mm curve, a 4 mm curve, a 3 mm curve, etc.) that can be deployed intraluminally to access the target site. In this embodiment, the introducer sheath may have a side port proximal to or along the pre-shaped curved portion through which the end effector 114 can be deployed. In other embodiments, the introducer sheath may be made from a rigid material, such as a metal material coated with an insulative or dielectric material. In this embodiment, the introducer sheath may be substantially straight and used to deliver the end effector 114 to the target site via a substantially straight pathway, such as through the middle meatus MM.

Image guidance may be used to aid the surgeon's positioning and manipulation of the distal portion of the shaft 116, as well as the deployment and manipulation of the end effector 114, specifically the first and second segments thereof. For example, an endoscope 100 and/or other visualization device can be positioned to visualize the target site, the positioning of the end effector 114 at the target site, and/or the end effector 114 during therapeutic neuromodulation. The endoscope 100 may be delivered proximate to the target site by extending through the nasal passage NP and through the middle meatus MM between the inferior and middle turbinates IT and MT. From the visualization location within the middle meatus MM, the endoscope 100 can be used to visualize the treatment site, surrounding regions of the nasal anatomy, and the end effector 114.

In some embodiments, the distal portion of the shaft 116 may be delivered via a working channel extending through an endoscope, and therefore the endoscope can provide direct in-line visualization of the target site and the end effector 114. In other embodiments, an endoscope is incorporated with the end effector 114 and/or the distal portion of the shaft 116 to provide in-line visualization of the end effector 114 and/or the surrounding nasal anatomy. In other embodiments, image guidance can be provided with various other guidance modalities, such as image filtering in the infrared (IR) spectrum to visualize the vasculature and/or other anatomical structures, computed tomography (CT), fluoroscopy, ultrasound, optical coherence tomography (OCT), and/or combinations thereof. Yet still, in some embodiments, image guidance components may be integrated with the neuromodulation device 102 to provide image guidance during positioning of the end effector 114.

Once positioned at the target site, the end effector may deliver the treatment energy at that targeted tissue, which can effectively create targeted micro-lesions in targeted neural tissue (including SPG) to thereby interrupt associated neural signals from passing therethrough. In particular, the targeted micro-lesions may cause multiple points of interruption of postganglionic parasympathetic fibers and trigeminal afferent branches to thereby reduce the frequency and intensity of headache disorder-related symptoms. The sphenopalatine ganglion (SPG) has been implicated in many headache disorders. The SPG lies behind the middle turbinate in the pterygopalatine fossa and contains a large cluster of sympathetic, parasympathetic, and sensory neurons that lies within a small pyramidal space. It is thought that headaches and facial pain originate from the activation of parasympathetic pathways within SPG. Irritation of the SPG causes cerebral vasodilatation, increases cerebral blood flow, and in turn releases acetylcholine (which can activate nociceptive fibers), and further releases nitric oxide, which ultimately cause headaches. Accordingly, multipoint lesion creation in the SPG region could result in multipoint interruption of postganglionic parasympathetic fibers and trigeminal afferent branches and reduce the frequency and intensity of headache symptoms.

Accordingly, the systems and methods of the present invention allow for accurate, minimally invasive, and localized application of energy to one or more target sites within the sino-nasal cavity to disrupt neural signals associated with nerves that play a role in headache disorders. By treating the underlying cause of headache disorders, the present invention is able to better address the symptoms without the inherent risks and drawbacks associated with pharmacotherapy and surgical treatments.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

1. A method for treating a headache disorder of a patient, the method comprising:
delivering treatment energy to one or more tissues at one or more target sites within a sino-nasal cavity of the patent to thereby cause multipoint interruption and/or multipoint modulation of one or more targeted neural tissues for the treatment of a headache disorder.

2. The method of claim 1, wherein the targeted neural tissue is associated with at least one of anterior ethmoidal nerve (AEN) tissue, posterior nasal nerve (PNN) tissue, and sphenopalatine ganglion (SPG) nerve cells.

3. The method of claim 2, wherein delivery of treatment energy creates targeted micro-lesions in targeted neural tissue to thereby interrupt associated neural signals.

4. The method of claim 3, wherein the targeted micro-lesions cause multiple points of interruption of postganglionic parasympathetic fibers and trigeminal afferent branches to thereby reduce the frequency and intensity of headache disorder-related symptoms.

5. The method of claim 3, wherein the targeted micro-lesions cause multiple points of interruption of nociceptive C fibers to thereby reduce the frequency and intensity of headache disorder-related symptoms.

6. The method of claim 3, wherein the targeted micro-lesions cause multiple points of interruption of accessible trigeminal branches associated with PNN tissue to thereby reduce the frequency and intensity of headache disorder-related symptoms.

7. The method of claim 3, wherein the targeted micro-lesions cause multiple points of interruption of AEN tissue to thereby reduce the frequency and intensity of headache disorder-related symptoms.

8. The method of claim 7, wherein the treatment energy is delivered at a level sufficient to create the targeted micro-lesions in the AEN tissue without damaging olfactory nerves to thereby preserve olfactory function.

9. The method of claim 2, wherein delivery of treatment energy results in modulation of nociceptive C fibers to thereby reduce the frequency and intensity of headache disorder-related symptoms.

10. The method of claim 1, wherein the headache disorder is selected from the group consisting of migraine, tension-type headache (TTH), cluster headache (CH) or trigeminal neuralgia, and medication-overuse headache (MOH).

11. The method of claim 1, wherein the multipoint interruption is accomplished by ablating neural tissue.

12. The method of claim 11, wherein the ablation is thermal ablation.

13. The method of claim 12, wherein the thermal ablation is cyro-ablation.

14. The method of claim 11, wherein the ablation is caused by delivery of radio-frequency (RF) energy.

15. The method of claim 1, wherein the treatment energy is delivered by a device comprising an end effector comprising at least one retractable and expandable segment.

16. The method of claim 15, wherein the end effector comprises a plurality of energy delivery elements provided on the at least one segment.

17. The method of claim 16, wherein, when in an expanded, deployed state, the at least one segment positions one or more of the plurality of energy delivery elements relative to the one or more target sites.

18. The method of claim 15, wherein the at least one retractable and expandable segment comprises a plurality of deformable wires and the plurality of energy delivery elements are operably associated with the plurality of deformable wires.

19. The method of claim 18, wherein the deformable wires comprise shape memory material.

20. The method of claim 15, wherein the end effector is a multi-segment end effector comprising at least a first retractable and expandable segment and a separate and distinct second retractable and expandable segment.
